(19) United States
(12) Patent Application Publication
(10) Pub. No.: US 2022/0152110 A1
(43) Pub. Date: May 19, 2022

(54) Switchable Chimeric Antigen Receptor-Engineered Human Natural Killer Cells

(71) Applicants: The Regents of the University of California, Oakland, CA (US); The Scripps Research Institute, La Jolla, CA (US)

(72) Inventors: Dan S. Kaufman, La Jolla, CA (US); Xiao-Hua Li, La Jolla, CA (US); Eduardo Laborda, La Jolla, CA (US); Travis Young, La Jolla, CA (US)

(21) Appl. No.: 17/441,653

(22) PCT Filed: Mar. 23, 2020
(86) PCT No.: PCT/US2020/024187

§ 371 Date: Sep. 21, 2021

Related U.S. Application Data
(60) Provisional application No. 62/822,389, filed on Mar. 22, 2019.

(51) Int. Cl. A61K 35/17; C07K 14/725; C07K 16/30; C07K 14/705; C07K 16/28; A61P 35/00

(52) U.S. Cl. CPC A61K 35/17; C07K 14/7051; C07K 16/30; C07K 14/7056; C07K 2319/03; A61P 35/00; C07K 2317/622; C07K 2317/55; C07K 16/2803

(57) ABSTRACT

Engineered natural killer cells with switchable chimeric antigen receptor, methods of manufacture, pharmaceutical compositions, and methods of use in treating cancer and viral infection.

### CROSS REFERENCE TO RELATED APPLICATIONS

This application claims the priority benefit of U.S. Provisional Application No. 62/822,389, filed Mar. 22, 2019, which application is incorporated herein by reference.

### TECHNICAL FIELD

The present invention relates to engineered immunotherapies.

### BACKGROUND

The existing CAR-engineered T cell-based (CAR-T) therapy represents one of the most successful immunotherapy approaches developed in recent years. Most CAR-T cell therapy has been used clinically to treat hematological malignancies by targeting the B cell-specific antigen, CD19. However, this approach is not without limitations, owing to some of the intrinsic properties of T cells, including alloreactivity that limits its use strictly to autologous patients, and side effects caused by uncontrolled proliferation or uncontrolled cytokine release. NK cells, on the other hand, function as allogenic cytotoxic effector cells that do not have to be applied on a patient specific basis, do not need prior sensitization, and are proved to be less toxic. For these reasons, CAR-engineered NK (CAR-NK) cells have increasingly attracted interests as an alternative CAR-cell therapy. However, there exists other unmet challenges. Targeting CAR-based therapies against solid tumors has been challenging due to the lack of truly tumor-specific antigens, as most targets are shared by non-malignant cells and can cause toxicity due to “on-target, off-tumor” effects.” A fine-tunable CAR therapy is useful to better identify and target tumors while limiting this toxicity. A second challenge is the difficult of scaling up CAR-cell manufacturing rate to meet patient's needs. This is because the conventional CAR-cells are made in a rigid form on a one-CAR for one-target basis. The preparation of each cell batch with one specificity demands time, cost, and sophisticated manufactural facilities, which often renders the treatment impossible for most patients. The recently developed sCAR, that uncouples effector cell activation functions from antigen recognition function is designed to overcome these hurdles.

One key goal of adoptive cell therapy is to precisely control the anti-tumor activity of the therapeutic cell population. Current strategies such as the FDA approved anti-CD19 CAR-T cells (tisagenlecleucel (Kymriah) and axicabtagene ciloleucel (Yescarta)) have been very effective, but lead to long-term (perhaps permanent) B cell aplasia and hypogammaglobulinemia that render patients with significant immunosuppression and susceptibility to infections^{1,2}. Additionally, toxicities such as CAR-mediated cytokine release syndrome and neurotoxicity can be difficult to control and lead to significant morbidity and even mortality in some cases¹⁻³. Many tumor antigens targeted by CARs can result in “on-target, off-tumor” toxicity, as has been well reviewed^{1,2}. For example, targeting of carcinoembryonic antigen by CAR-T cells in patients with colon cancer resulted in severe colitis due to antigen recognition of normal colonic tissue⁴. Unfortunately, treatment of a patient with anti-Her2 CAR-T cells lead to death, likely due to Her2 expression on pulmonary cells⁵. In the same way CARs against AML antigens are also problematic as most are also shared by normal hematopoietic stem cells, potentially resulting in prolonged bone marrow aplasia⁶⁻⁸. Control of CAR-T cell activity has been proposed using options such as suicide switches and RNA-based CAR-expression^{1,9-11} However, kill switches do not provide control over T-cell activation and expansion, and result in the irreversible elimination of potentially therapeutic CAR-T cells. RNA-based systems lead to only transient CAR-expression, less anti-tumor activity, and do not capture a fully dynamic and titratable on/off activity^{9,10}.

Previous studies using sCAR-T cells demonstrate selective formation of immunological synapses in which the sCAR-T cell, the switch, and the target cell all interact in a structurally defined and temporally controlled manner. This sCAR-T cell system has demonstrated potent killing of CD19+ cell B cell malignancies, including production of a T central memory (Tcm) population that enables effective re-dosing of the soluble switch component (only a single dose of sCAR-T cells is administered) to treat relapsed disease^{12,13} The use of a soluble switch also enables precise titration of CAR engagement. For example, patients can first be treated with low dose of the switch to minimize CRS or neurotoxicity, then with higher subsequent doses to increase anti-tumor activity. Additionally, this system easily enables use of more than one switch component to enable dual targeting of tumor cell antigens to prevent relapse due to loss of a single tumor antigen^{12,13}. To date, pre-clinical studies demonstrate these sCAR-T cells cannot only target and killed CD19⁺ B cell malignancies, but also breast cancer and pancreatic cancer with an anti-Her2 switch¹²⁻¹⁵.

Finally, many other CAR-based systems such as the Syn-Notch system, “conditional CARs”, other switch-mediated CARs, as well as other systems have been proposed to improve targeting of solid tumors while maintaining safety and control over treatment^{2,16-18}.

### SUMMARY OF THE INVENTION

The disclosure provides a natural killer (NK) cell engineered with switchable chimeric antigen receptor (sCAR), method for the manufacture thereof, and methods of use.

In embodiments, the present invention provides a natural killer (NK) cell engineered with a switchable chimeric antigen receptor (sCAR). In embodiments, the sCAR comprises an antibody scFv region specific for binding to a peptide neoantigen epitope (PNE). In embodiments, the sCAR further comprises an NKG2D transmembrane domain, 2B4 co-stimulatory domain, and/or CD3ζ chain (or mutations thereof). In embodiments, the NK cell further comprises a switch bound to the sCAR, wherein the switch comprises a peptide neoantigen epitope (PNE) fused to an anti-cancer or anti-virus antibody Fab region specific for binding to a cancer antigen or virus antigen. In embodiments, the cancer antigen is CD19 or Frizzled 7. In embodiments, the invention provides that the cancer or viral antigens can be any of those disclosed herein or known in the art. In embodiments, the NK cell is derived from a human induced pluripotent cell.

In embodiments, the present invention provides a method of treating a cancer or a virus in a subject comprising administering to a subject in need thereof an effective amount of a natural killer (NK) cell engineered with a switchable chimeric antigen receptor (sCAR) activated against an antigen of the cancer or the virus.

In embodiments, the sCAR comprises an antibody scFv region specific for binding to a peptide neoantigen epitope (PNE). In embodiments, the sCAR further comprises an NKG2D transmembrane domain, 2B4 co-stimulatory domain, and CD3ζ chain.

In embodiments, the sCAR is further activated by being bound to a switch, wherein the switch comprises a PNE fused to an anti-cancer or anti-virus antibody Fab region specific for binding to the cancer antigen or virus antigen, respectively. In embodiments, the cancer antigen is CD19 or Frizzled 7. In embodiments, the invention provides that the cancer or viral antigens can be any of those disclosed herein or known in the art.

In embodiments, the NK cell is allogenic. In embodiments, the cancer is refractory. In embodiments the cancer is hemotologic or a solid tumor. In embodiments, the tumor is lymphatic or ovarian. In embodiments, the invention provides that the cancer can be any of the cancers disclosed or known. In embodiments, the invention provides that the virus can be any of the viruses disclosed or known.

In embodiments, the invention provides that the methods can further comprise administration of a therapeutic amount of monoclonal antibody therapy against the cancer or virus.

In embodiments, the invention provides pharmaceutical compositions comprising the NK cells as described herein. In embodiments, the invention provides a cell culture of the sCAR-NK cells.

In embodiments, the invention provides a method of manufacturing a natural killer (NK) cell, comprising: engineering a NK cell to display a transmembrane protein comprising a switchable chimeric antigen receptor (sCAR); and storing the engineered NK cell for later activation of the sCAR.

In embodiments, the invention provides the sCAR comprises an antibody scFv region specific for binding to a peptide neoantigen epitope (PNE). In embodiments, the sCAR further comprises an NKG2D transmembrane domain, 2B4 co-stimulatory domain, and CD3ζ chain.

In embodiments, the invention provides that the methods of manufacture further comprise activating the sCAR by binding the sCAR to a switch, wherein the switch comprises a PNE fused to an anti-cancer or anti-virus antibody Fab region specific for binding to a cancer antigen or virus region, respectively.

### BRIEF DESCRIPTION OF THE DRAWINGS

FIGS. 1A-1C show a schematic comparison of conventional CAR and sCAR.

FIGS. 2A-2B show a sCAR mediated anti-tumor activity using NK92 effector cells.

FIGS. 3A-3B show hematopoietic and NK cell differentiation in sCAR-expressing iPSCs.

FIG. 4 shows sCAR mediated anti-tumor activity in iPSC-NK cells.

FIG. 5 shows an IncuCyte killing assay.

FIG. 6 shows an in vitro killing assay with artificially mixed cell lines.

FIG. 7 shows configurations of different switches used throughout.

FIG. 8 shows CAR4-NK92-induced killing of Nalm6 in the presence of the increased concentrations of anti-CD19 switches.

FIGS. 9A-9C show antigen expression of AML antigens on the target cells and switch-mediated killing assay on AML cell lines.

FIGS. 10A-10C are a demonstration of Antigen specificity of switch-mediated killing in PBMC with a naturally mixed cell population.

FIG. 11 is a comparison of sCAR4 to the conventional CD19-CAR4.

FIG. 12 shows GFP and sCAR4 expression on NK92 cells transfected with either SB-sCAR4-P2A-GFP or SB-sCAR4-IRES-GFP.

FIG. 13 shows a comparison of NK92-sCAR-IRES and NK92-P2A-GFP in an in vitro coculture killing assay in which MA148 cells were cocultured with either WT NK92 or NK92 transfected with NK92-sCAR-IRES or NK92-P2A-GFP in the presence of increasing levels of anti-Fzd7 or control switches.

FIG. 14 shows expression of GFP and sCAR4 on the surface of transfected iPSCs.

FIGS. 15A-15B show regeneration of iPSC-derived NK cells expressing sCAR4-P2A-GFP.

### DETAILED DESCRIPTION

The present invention relates to the engineering of natural killer (NK) cells with a switchable chimeric antigen receptor (sCAR) that is designed to create a targeted, NK cell-based therapeutic modality, referred to as sCAR-NK cells, to more effectively treat refractory cancers—both hematologic malignancies and solid tumors. Specifically, by equipping NK cell with the sCAR, it enables NK cells to target tumors in a tightly titratable manner, as well as target multiple tumor antigens simultaneously or sequentially. Therefore, this system mediates improved anti-tumor killing, prevents development of tumor antigen loss as a means of tumor escape from immune-mediated killing, and minimizes toxicity to the patient.

In the invention, different technologies are combined in a unique way to first make a NK cell-specific sCAR, and then genetically engineer NK cells with NK-sCAR to create the unique sCAR-NK cells as a potential therapeutic cell product. This new type of cell integrates the best traits of CAR-T cells and NK cells to mediate better efficacy, versatility, dose-control, and minimal toxicity to the normal cells. In embodiments, NK92 cells, a clinically used NK cell line are used, as well as NK cells derived from human induced pluripotent stem cells (iPSC-NK) to demonstrate the protypes of the sCAR-NK cells. However, the invention includes the use of other types of NK cells, including NK cells generated from other stem cell types, or isolated or produced from peripheral blood or umbilical cord blood.

The invention improves anti-tumor activity and safety of NK cell-mediated immunotherapy by use of a novel recombinant antibody-based bifunctional switch system that consists of a tumor antigen-specific Fab molecule fused to a peptide neo-epitope (PNE), which is recognized exclusively by a PNE-specific switchable CAR (sCAR).

Additional data demonstrate effective use of an anti-FZD7 switch (to target solid tumors) and an anti-CLL1 switch (for AML). Therefore, this system has wide applicability for both hematologic malignancies and solid tumors. Although the focus of this invention is the targeting and treatment of cancerous cells, viral infections may also be treated using this invention with viral antigen instead of cancer antigen.

The present switchable CAR system combined with sCAR-expressing iPSC-NK cells is the most feasible and widely applicable strategy to readily translate into effective patient therapies.

Existing art includes “conventional” CAR-T cells, sCAR-expressing T cells and CAR-NK cells. sCAR-NK cells improve upon these approaches by working as allogeneic effector cells that do not have to be patient matched (as is the case for conventional CAR-T cells and sCAR-T cells). Additionally, this invention improves upon other CAR-NK cells by allowing maximal flexibility in targeting. The sCAR system is combined with iPSC-derived CAR-NK cells. This combination offers maximum flexibility to utilize one standardized allogeneic effector cell population combined with the soluble switches to create a universal cell therapy approach—both because the NK cells do not need to be derived from individual patients and because the soluble switches can be used to target essentially any tumor antigen (or multiple tumor antigens) without the need to engineer a new effector cell population.

Additionally, tumor antigen loss escape variants can be prevented with sCAR-iPSC-NK cells combined with switches against 2 (or more) tumor antigens. Switch-mediated targeting can also be combined with therapeutic monoclonal antibodies (anti-Her2, anti-EGFR, etc) to more effectively target and kill tumors.

This invention allows the use of the same sCAR-expressing iPSC-derived NK cells for treatment of both hematological malignancies and solid tumors. In this manner, sCAR-iPSC-NK cells again provide a universal strategy for an “off-the-shelf” cellular immunotherapy that can lead to a paradigm-shifting impact in the field.

In embodiments, the present invention provides a natural killer (NK) cell engineered with a switchable chimeric antigen receptor (sCAR). In embodiments, the sCAR comprises an antibody scFv region specific for binding to a peptide neoantigen epitope (PNE). In embodiments, the sCAR further comprises an NKG2D transmembrane domain, 2B4 co-stimulatory domain, and CD3ζ chain. In embodiments, the sCAR comprises alternative signaling domains including, but not limited to: extracellular domain of CD8a extracellular domain, transmembrane domain of CD28, CD16, NKp44, NKp46; cytoplasmic signaling domain of CD28, CD137, DAP10, and DAP12.⁽¹⁹⁾

In embodiments, the NK cell further comprises a switch bound to the sCAR, wherein the switch comprises a PNE fused to an anti-cancer or anti-virus antibody Fab region specific for binding to a cancer antigen or virus antigen. In embodiments, the cancer antigen is CD19 or Frizzled 7. In embodiments, the invention provides that the cancer or viral antigens can be any of those disclosed herein or known in the art.

In embodiments, the NK cell is derived from a human induced pluripotent cell. In embodiments, the invention provides a cell culture of the sCAR-NK cells.

In embodiments, the present invention provides a method of treating a cancer or a virus in a subject comprising administering to a subject in need thereof an effective amount of a natural killer (NK) cell engineered with a switchable chimeric antigen receptor (sCAR) activated against an antigen of the cancer or the virus.

In embodiments, the sCAR comprises an antibody scFv region specific for binding to a peptide neoantigen epitope (PNE). In embodiments, the sCAR further comprises an NKG2D transmembrane domain, 2B4 co-stimulatory domain, and CD3ζ chain.

In embodiments, the sCAR-NK cell is further activated by being bound to a switch, wherein the switch comprises a PNE fused to an anti-cancer or anti-virus antibody Fab region specific for binding to the cancer antigen or virus antigen, respectively. In embodiments, the cancer antigen is CD19 or Frizzled 7. In embodiments, the invention provides that the cancer or viral antigens can be any of those disclosed herein or known in the art.

In embodiments, the switch can use a binding molecule other than a Fab fragment targeting a cancer or viral antigen. In embodiments, natural antigen-interacting protein domains are used to bind to a specific antigen.⁽²⁶⁾ In embodiments, proteins naturally express activating receptors, which, upon ligand binding, activate an immune response. In embodiments, NK cells express natural killer group 2, member D (NKG2D), an activating receptor, which upon ligand binding, activates immune cells through the adaptor molecule DAP10, thereby triggering cellular proliferation, pro-inflammatory cytokine production, and target cell elimination. NKG2D ligands (NKG2DLs) include major histocompatibility complex (MHC) class I-related chain A and B (MICA and MICB, respectively) and six unique long 16 binding protein (ULBP1-6). In embodiments, other NK cell activating receptors may be used. Examples of other NK cell activating receptors, include: natural cytotoxic receptors (NCR), DNAX accessory molecule-1 (DNAM1) and activating killer cell immunoglobulin-like receptors (KAR).

In embodiments, the NK cell is allogenic. In embodiments, the cancer is refractory. In embodiments the cancer is hematologic or a solid tumor. In embodiments, the tumor is lymphatic or ovarian. In embodiments, the invention provides that the cancer can be any of the cancers disclosed or known. In embodiments, the invention provides that the virus can be any of the viruses disclosed or known.

In embodiments, the invention provides that the methods can further comprise administration of a therapeutic amount of monoclonal antibody therapy against the cancer or virus.

In embodiments, the invention provides pharmaceutical compositions comprising the NK cells as described herein. In embodiments, the invention provides a cell culture of the sCAR-NK cells.

In embodiments, the invention provides methods of manufacturing a natural killer sCAR-NK cell, comprising: engineering a NK cell to display a transmembrane protein comprising a switchable chimeric antigen receptor (sCAR); and storing the engineered NK cell for later activation of the sCAR.

In embodiments, the invention provides the sCAR comprises an antibody scFv region specific for binding to a peptide neoantigen epitope (PNE). In embodiments, the sCAR further comprises an NKG2D transmembrane domain, 2B4 co-stimulatory domain, and CD3ζ chain.

In embodiments, the invention provides that the methods of manufacture further comprise activating the sCAR by binding the sCAR to a switch, wherein the switch comprises a PNE fused to an anti-cancer or anti-virus antibody Fab region specific for binding to a cancer antigen or virus region, respectively.

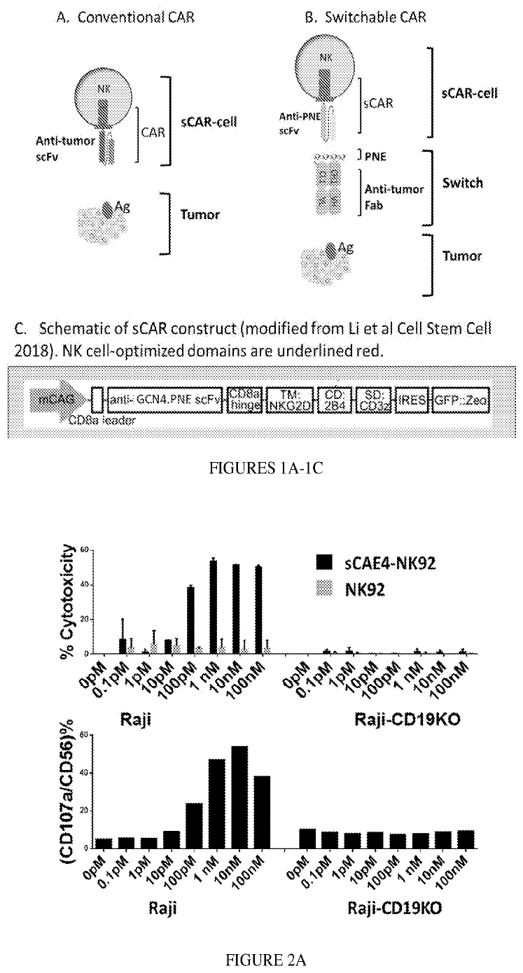

An existing sCAR module was adopted and engineered into NK cells, including NK92 and iPS-derived NK cells (iPS-NKs). As shown in FIGS. 1A-1C, compared with the conventional CARs (FIG. 1A) which is composed of a tumor antigen recognition ectodomain (svFc) and an intracellular activation domain fused together through a hinge and a transmembrane domain, sCAR (FIG. 1B) is different because in general it is composed of a similar domain display except that the specificity of the svFc is not directed to a tumor antigen but to a generic peptide of 14 aa derived from the yeast GCN4 protein, namely peptide neoantigen epitope (PNE). The PNE, together with its fused fragment of a monoclonal antibody (Fab), serves as a “switch” molecule that determines antigen specificity. By having this flexible switch, sCAR system allows the preparation of CAR-cells to be generic and independent on which tumor antigen to target. In addition, it allows redirection of antigen target even after the sCAR-cells are already administrated in the patient. Also, the switch-dependence allows for more precise control over activity of the CAR and it is predicted that this control will reduce current complications of CAR therapy by proper dosing. Furthermore, as already briefly mentioned, in order to better promote NK-cell cytotoxic effects to tumor cells, the sCAR that was constructed in this invention contains a PNE-specific scFv that is then combined with NK cell-optimized CAR4 signaling motifs consisting of the NKG2D transmembrane domain, 2B4 co-stimulatory domain and the CD3ζ chain (referred as CAR4, FIG. 1C). The incorporation of these motifs into the sCAR system will further enhance NK cell functions.

Both NK92 cells and iPSC-derived NK cells for production of sCAR-expressing NK cells have been used. There are several advantages of using iPSC-derived NK cells. These cells have normal NK cell phenotype and gene expression profile (while NK92 cells are aneuploid and must be irradiated before administering to patients). Production of iPSC-derived NK cells can now be done under cGMP conditions at clinical scale. Therefore, sCAR-expressing iPSC-derived NK cells provide a uniform population that can be produced in essentially unlimited supply. As NK cells do not have to be matched to a specific patient (i.e. they function as allogeneic effector cells) one standardized population of sCAR-expressing iPSC-derived NK cells combined with soluble anti-tumor switches can be used to target different tumors from one standardized “off-the-shelf” NK cell product. This provides a “universal” approach to targeted cell-based therapies. Additionally, more than one tumor antigen can be targeted by using multiple switches with the same sCAR-expressing NK cells.

The invention may be applied commercially as: (1) A therapeutic modality for cancer as described in the invention; (2) A therapeutic modality for infectious disease (by targeting antigens on virally infected such as gp120 on HIV-infected cells). Although the descriptions and the antigens of choice in our proof-of-concept studies are based on cancers, the utility of the invention can be easily transferred to infectious diseases, especially viral infections, as the mechanisms of NK cells to kill cancerous cells and virally infected cells are similar; and (3) A tool for research on antigen discovery and validation. The future success of CAR-based cancer immunotherapy depends heavily on discovery of reliable cancer-specific antigens. Current advances in genomics, surface proteomics, and bioinformatics make it possible to discover surface cancer antigens in a much more rapid speed than ever before. Relying on the conventional CAR therapy, it would be impossible to accommodate the ever-growing demand for validating CAR therapies with a large scale of antigen candidates. This invention of sCAR-NK cells provides a truly universal approach for cell-based immunotherapies.

Potential targets using the sCAR-NK system include, but are not limited to, targets for solid tumors, hematological malignancies, and viral infections.

Examples of switch targets for solid tumors include, but are not limited to: AChR (Fetal acetylcholine receptor), B7-H4, CAIX (carbonic anhydrase IX), CD133 (prominin-1), CD44v6, CD47 (integrin associated protein or IAP), CD70—used in multiple disease categories, CEA (carcinoembryonic antigen), c-Met (c-mesenchymal-epithelial transition factor), DLL3 (Delta-like 3), EGFR (epidermal growth factor receptor), EGFRvIII (type III variant epidermal growth factor receptor, EpCAM (epithelial cell adhesion molecule), EphA2 (Erythropoetin producing hepatocellular carcinoma A2), ErbB2, FAP (fibroblast activation protein), FRa (folate receptor alpha), Frizzled 7 (Fzd7), GD2 (Ganglioside GD2), GPC3 (Glypican-3), GUCY2C (Guanylyl cyclase C), HER1 (human epidermal growth factor receptor 1), HER2 (ErbB2, human epidermal growth factor 2), ICAM-1 (intercellular adhesion molecule 1), IL11Rα (interleukin 11 receptor a), IL13Rα2 (interleukin 13 receptor a2), L1-CAM (human L1 cell adhesion molecule, CD171), LeY (Lewis Y antigen), MAGE (Melanoma-associated antigen), MCAM (CD146) (melanoma cell adhesion molecule), Mesothelin, MUC1 (mucin 1), MUC16 ecto (mucin 16), NKG2DLs (natural killer group 2 member D ligands), NY-ESO-1 (Cancer/testis antigen 1), PD-L1 (B7-H1) (CD274), PSCA (prostate stem cells antigen), PSMA (prostate-specific membrane antigen), ROR1 (receptor tyrosine kinase-like orphan receptor)—used in multiple disease categories, TAG72 (tumor-associated glycoproteins-72), and VEGFR (Vascular endothelial growth factor receptor 1).

Examples of switch targets for hematological malignancies include, but are not limited to: BCMA (B-cell maturation antigen), CD123, CD138 (syndecan-1), CD19, CD20, CD22, CD24, CD30, CD33, CD37, CD38, CD4—used in multiple disease categories, CD7, CD70—used in multiple disease categories, CLL1, CS1 (connecting segment 1), kappa light chain, and ROR1 (receptor tyrosine kinase-like orphan receptor)—used in multiple disease categories.

Examples of switch targets for viral infections include, but are not limited to: HIV (Envelop glycoproteins gp120), CD4—used in multiple disease categories, HBV (HBsAg—Hepatitis B surface antigen), EBV (LMP1—latent membrane protein 1), CMV (gB—glycoprotein B), and HCV (Glycoprotein E2).

All publications, patents, and patent applications mentioned in this specification are herein incorporated by reference to the same extent as if each individual publication, patent, or patent application was specifically and individually indicated to be incorporated by reference.

Unless defined otherwise, all technical and scientific terms and any acronyms used herein have the same meanings as commonly understood by one of ordinary skill in the art in the field of the invention. Although any methods and materials similar or equivalent to those described herein can be used in the practice of the present invention, the exemplary methods, devices, and materials are described herein.

The practice of the present invention will employ, unless otherwise indicated, conventional techniques of molecular biology (including recombinant techniques), microbiology, cell biology, biochemistry and immunology, which are within the skill of the art. Such techniques are explained fully in the literature, such as, Molecular Cloning: A Laboratory Manual, 2^{nd} ed. (Sambrook et al., 1989); Oligonucleotide Synthesis (M. J. Gait, ed., 1984); Animal Cell Culture (R. I. Freshney, ed., 1987); Methods in Enzymology (Academic Press, Inc.); Current Protocols in Molecular Biology (F. M. Ausubel et al., eds., 1987, and periodic updates); PCR: The Polymerase Chain Reaction (Mullis et al., eds., 1994); Remington, The Science and Practice of Pharmacy, 20^{th} ed., (Lippincott, Williams & Wilkins 2003), and Remington, The Science and Practice of Pharmacy, 22^{th} ed., (Pharmaceutical Press and Philadelphia College of Pharmacy at University of the Sciences 2012).

As used herein, the terms “comprises,” “comprising,” “includes,” “including,” “has,” “having,” “contains”, “containing,” “characterized by,” or any other variation thereof, are intended to encompass a non-exclusive inclusion, subject to any limitation explicitly indicated otherwise, of the recited components. For example, a fusion protein, a pharmaceutical composition, and/or a method that “comprises” a list of elements (e.g., components, features, or steps) is not necessarily limited to only those elements (or components or steps), but may include other elements (or components or steps) not expressly listed or inherent to the fusion protein, pharmaceutical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a fusion protein, pharmaceutical composition, and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

When introducing elements of the present invention or the preferred embodiment(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “and/or” when used in a list of two or more items, means that any one of the listed items can be employed by itself or in combination with any one or more of the listed items. For example, the expression “A and/or B” is intended to mean either or both of A and B, i.e. A alone, B alone or A and B in combination. The expression “A, B and/or C” is intended to mean A alone, B alone, C alone, A and B in combination, A and C in combination, B and C in combination or A, B, and C in combination.

It is understood that aspects and embodiments of the invention described herein include “consisting” and/or “consisting essentially of” aspects and embodiments.

It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range. Values or ranges may be also be expressed herein as “about,” from “about” one particular value, and/or to “about” another particular value. When such values or ranges are expressed, other embodiments disclosed include the specific value recited, from the one particular value, and/or to the other particular value. Similarly, when values are expressed as approximations, by use of the antecedent “about,” it will be understood that the particular value forms another embodiment. It will be further understood that there are a number of values disclosed therein, and that each value is also herein disclosed as “about” that particular value in addition to the value itself. In embodiments, “about” can be used to mean, for example, within 10% of the recited value, within 5% of the recited value, or within 2% of the recited value.

As used herein, “patient” or “subject” means a human or animal subject to be treated.

As used herein the term “pharmaceutical composition” refers to a pharmaceutical acceptable compositions, wherein the composition comprises a pharmaceutically active agent, and in some embodiments further comprises a pharmaceutically acceptable carrier. In some embodiments, the pharmaceutical composition may be a combination of pharmaceutically active agents and carriers.

The term “combination” refers to either a fixed combination in one dosage unit form, or a kit of parts for the combined administration where one or more active compounds and a combination partner (e.g., another drug as explained below, also referred to as “therapeutic agent” or “co-agent”) may be administered independently at the same time or separately within time intervals. In some circumstances, the combination partners show a cooperative, e.g., synergistic effect. The terms “co-administration” or “combined administration” or the like as utilized herein are meant to encompass administration of the selected combination partner to a single subject in need thereof (e.g., a patient), and are intended to include treatment regimens in which the agents are not necessarily administered by the same route of administration or at the same time. The term “pharmaceutical combination” as used herein means a product that results from the mixing or combining of more than one active ingredient and includes both fixed and non-fixed combinations of the active ingredients. The term “fixed combination” means that the active ingredients, e.g., a compound and a combination partner, are both administered to a patient simultaneously in the form of a single entity or dosage. The term “non-fixed combination” means that the active ingredients, e.g., a compound and a combination partner, are both administered to a patient as separate entities either simultaneously, concurrently or sequentially with no specific time limits, wherein such administration provides therapeutically effective levels of the two compounds in the body of the patient. The latter also applies to cocktail therapy, e.g., the administration of three or more active ingredients.

As used herein the term “pharmaceutically acceptable” means approved by a regulatory agency of the Federal or a state government or listed in the U.S. Pharmacopoeia, other generally recognized pharmacopoeia in addition to other formulations that are safe for use in animals, and more particularly in humans and/or non-human mammals.

As used herein the term “pharmaceutically acceptable carrier” refers to an excipient, diluent, preservative, solubilizer, emulsifier, adjuvant, and/or vehicle with which demethylation compound(s), is administered. Such carriers may be sterile liquids, such as water and oils, including those of petroleum, animal, vegetable or synthetic origin, such as peanut oil, soybean oil, mineral oil, sesame oil and the like, polyethylene glycols, glycerine, propylene glycol or other synthetic solvents. Antibacterial agents such as benzyl alcohol or methyl parabens; antioxidants such as ascorbic acid or sodium bisulfite; chelating agents such as ethylenediaminetetraacetic acid; and agents for the adjustment of tonicity such as sodium chloride or dextrose may also be a carrier. Methods for producing compositions in combination with carriers are known to those of skill in the art. In some embodiments, the language “pharmaceutically acceptable carrier” is intended to include any and all solvents, dispersion media, coatings, isotonic and absorption delaying agents, and the like, compatible with pharmaceutical administration. The use of such media and agents for pharmaceutically active substances is well known in the art. See, e.g., Remington, The Science and Practice of Pharmacy, 20th ed., (Lippincott, Williams & Wilkins 2003). Except insofar as any conventional media or agent is incompatible with the active compound, such use in the compositions is contemplated.

As used herein, “therapeutically effective” refers to an amount of a pharmaceutically active compound(s) that is sufficient to treat or ameliorate, or in some manner reduce the symptoms associated with diseases and medical conditions. When used with reference to a method, the method is sufficiently effective to treat or ameliorate, or in some manner reduce the symptoms associated with diseases or conditions. For example, an effective amount in reference to diseases is that amount which is sufficient to block or prevent onset; or if disease pathology has begun, to palliate, ameliorate, stabilize, reverse or slow progression of the disease, or otherwise reduce pathological consequences of the disease. In any case, an effective amount may be given in single or divided doses.

As used herein, the terms “treat,” “treatment,” or “treating” embraces at least an amelioration of the symptoms associated with diseases in the patient, where amelioration is used in a broad sense to refer to at least a reduction in the magnitude of a parameter, e.g. a symptom associated with the disease or condition being treated. As such, “treatment” also includes situations where the disease, disorder, or pathological condition, or at least symptoms associated therewith, are completely inhibited (e.g. prevented from happening) or stopped (e.g. terminated) such that the patient no longer suffers from the condition, or at least the symptoms that characterize the condition.

As used herein, and unless otherwise specified, the terms “prevent,” “preventing” and “prevention” refer to the prevention of the onset, recurrence or spread of a disease or disorder, or of one or more symptoms thereof. In certain embodiments, the terms refer to the treatment with or administration of a compound or dosage form provided herein, with or without one or more other additional active agent(s), prior to the onset of symptoms, particularly to subjects at risk of disease or disorders provided herein. The terms encompass the inhibition or reduction of a symptom of the particular disease. In certain embodiments, subjects with familial history of a disease are potential candidates for preventive regimens. In certain embodiments, subjects who have a history of recurring symptoms are also potential candidates for prevention. In this regard, the term “prevention” may be interchangeably used with the term “prophylactic treatment.”

As used herein, and unless otherwise specified, a “prophylactically effective amount” of a compound is an amount sufficient to prevent a disease or disorder, or prevent its recurrence. A prophylactically effective amount of a compound means an amount of therapeutic agent, alone or in combination with one or more other agent(s), which provides a prophylactic benefit in the prevention of the disease. The term “prophylactically effective amount” can encompass an amount that improves overall prophylaxis or enhances the prophylactic efficacy of another prophylactic agent. As used herein, and unless otherwise specified, the term “subject” is defined herein to include animals such as mammals, including, but not limited to, primates (e.g., humans), cows, sheep, goats, horses, dogs, cats, rabbits, rats, mice, and the like. In specific embodiments, the subject is a human. The terms “subject” and “patient” are used interchangeably herein in reference, for example, to a mammalian subject, such as a human.

As used herein, the term “antibody” refers to immunoglobulin molecules and immunologically active portions of immunoglobulin (Ig) molecules, i.e., molecules that contain an antigen binding site that immunologically binds an antigen. Antibodies include, but are not limited to, polyclonal, monoclonal, chimeric, dAb (domain antibody), single chain, F_{ab}, F_{ab′} and F_{(ab)2} fragments, single-chain Fv fragments (scFvs), and an F_{ab} expression library. The basic antibody structural unit is known to comprise a tetramer. Each tetramer is composed of two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. The carboxy-terminal portion of each chain defines a constant region primarily responsible for effector function. In general, antibody molecules obtained from humans relate to any of the classes IgG, IgM, IgA, IgE and IgD, which differ from one another by the nature of the heavy chain present in the molecule. Certain classes have subclasses as well, such as IgG₁, IgG₂, and others. Furthermore, in humans, the light chain may be a kappa chain or a lambda chain.

The term “antibody” as used herein encompasses monoclonal antibodies (including full length monoclonal antibodies), polyclonal antibodies, multi-specific antibodies (e.g., bi-specific antibodies), and antibody fragments so long as they exhibit the desired biological activity of binding to a target antigenic site and its isoforms of interest. The term “antibody fragments” comprise a portion of a full length antibody, generally the antigen binding or variable region thereof. Fc, Fv and Fab regions of antibodies are well-known in the art. The term “antibody” as used herein encompasses any antibodies derived from any species and resources, including but not limited to, human antibody, rat antibody, mouse antibody, rabbit antibody, and so on, and can be synthetically made or naturally-occurring.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations which typically include different antibodies directed against different determinants (epitopes), each monoclonal antibody is directed against a single determinant on the antigen. The “monoclonal antibodies” may also be isolated from phage antibody libraries using the techniques known in the art.

The monoclonal antibodies herein include “chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. As used herein, a “chimeric protein” or “fusion protein” comprises a first polypeptide operatively linked to a second polypeptide. Chimeric proteins may optionally comprise a third, fourth or fifth or other polypeptide operatively linked to a first or second polypeptide. Chimeric proteins may comprise two or more different polypeptides. Chimeric proteins may comprise multiple copies of the same polypeptide. Chimeric proteins may also comprise one or more mutations in one or more of the polypeptides. Methods for making chimeric proteins are well known in the art.

In order to avoid potential immunogenicity of the monoclonal antibodies in humans, the monoclonal antibodies that have the desired function are preferably human or humanized “Humanized” forms of non-human (e.g., murine) antibodies are chimeric antibodies that contain minimal sequence derived from non-human immunoglobulin. For the most part, humanized antibodies are human immunoglobulins (recipient antibody) in which hyper variable region residues of the recipient are replaced by hyper variable region residues from a non-human species (donor antibody) such as mouse, rat, rabbit or nonhuman primate having the desired specificity, affinity, and capacity. Furthermore, humanized antibodies may comprise residues that are not found in the recipient antibody or in the donor antibody. These modifications are made to further refine antibody performance.

The term “antigen-binding site” or “binding portion” refers to the part of the antibody molecule that participates in antigen binding. The antigen binding site is formed by amino acid residues of the N-terminal variable (“V”) regions of the heavy (“H”) and light (“L”) chains. Three highly divergent stretches within the V regions of the heavy and light chains, referred to as “hypervariable regions,” are interposed between more conserved flanking stretches known as “framework regions,” or “FRs”. Thus, the term “FR” refers to amino acid sequences which are naturally found between, and adjacent to, hypervariable regions in immunoglobulins. In an antibody molecule, the three hypervariable regions of a light chain and the three hypervariable regions of a heavy chain are disposed relative to each other in three dimensional space to form an antigen-binding surface. The antigen-binding surface is complementary to the three-dimensional surface of a bound antigen, and the three hypervariable regions of each of the heavy and light chains are referred to as “complementary-determining regions,” or “CDRs.” The assignment of amino acids to each domain is in accordance with the definitions of Kabat Sequences of Proteins of Immunological Interest (National Institutes of Health, Bethesda, Md. (1987 and 1991)), or Chothia & Lesk J. Mol. Biol. 196:901-917 (1987), Chothia et al. Nature 342:878-883 (1989). Guidelines for the identification of CDRs is available at http://www.bioinf.org.uk/abs/#cdrid.

As used herein, the term “epitope” includes any protein determinant of an antigen capable of specifically binding an antibody or a T-cell receptor. Epitopic determinants usually consist of chemically active surface groupings of molecules such as amino acids or sugar side chains and usually have specific three dimensional structural characteristics, as well as specific charge characteristics. For example, antibodies may be raised against N-terminal or C-terminal peptides of a polypeptide. An antibody is said to specifically bind an antigen when the dissociation constant is ≤1 μM; preferably ≤100 nM and most preferably ≤10 nM. As used herein, the term “peptide neoantigen epitope (PNE)” includes any epitope not previously recognized by the immune system. PNEs may be used to target tumor cells that have a tumor specific mutant antigen (neoantigen), allowing for individualized immunotherapy.

### Examples

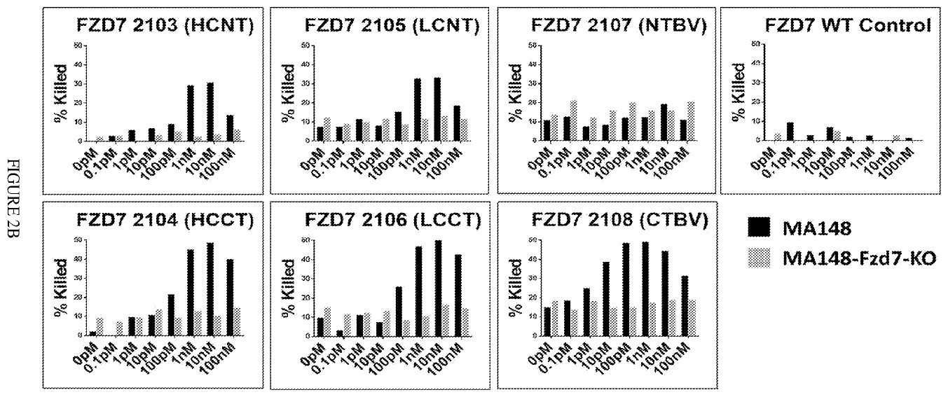

sCAR-expressing NK cells. Since the sCAR system had only been previously tested in T cells, an initial proof-of-concept testing of an anti-CD19 sCAR expressed in NK92 cells was performed. NK92 is a NK cell line that has been previously used to test novel NK cell-CAR constructs¹⁹. Here, sCAR was reengineered to include the CAR4 NK cell-signaling domains described above that mediate activation of NK cell intracellular signaling pathways and improve NK-CAR anti-tumor activity compared to CAR-T cell constructs expressed in NK cells¹⁹. The efficacy of both an anti-CD19 switch and anti-FZD7 switch to mediate specific killing of either CD19⁺ Raji B cell lymphoblastic leukemia (hematological malignancy model) or the FZD7⁺ MA148 ovarian cancer cell line (solid tumor model) was demonstrated (FIGS. 2A-2B). For the anti-CD19 switch, specificity of killing was demonstrated only when the anti-CD19 switch is present using CD19-deleted Raji cells and CD19-negative K562 cells (FIG. 2A and data not shown). FIG. 2B shows killing of MA148 cells by sCAR4-NK92 cells in the presence of different Fzd7 switches.

In the MA-148 model, the CRISPR/Cas9 system was again used to derive FZD7-negative (MA148K0 cells) to demonstrate specific FZD7 engagement. Interestingly, 5 out of 6 FZD7 switches combined with NK92-sCAR4 were able to mediate antigen-specific killing of MA148 cells, but not the FDZ7-negative MA148 KO cells. As expected, the WT switch-lacking PNE, did not bind to sCAR4 on NK92 cells and did not mediate killing of MA148 cells.

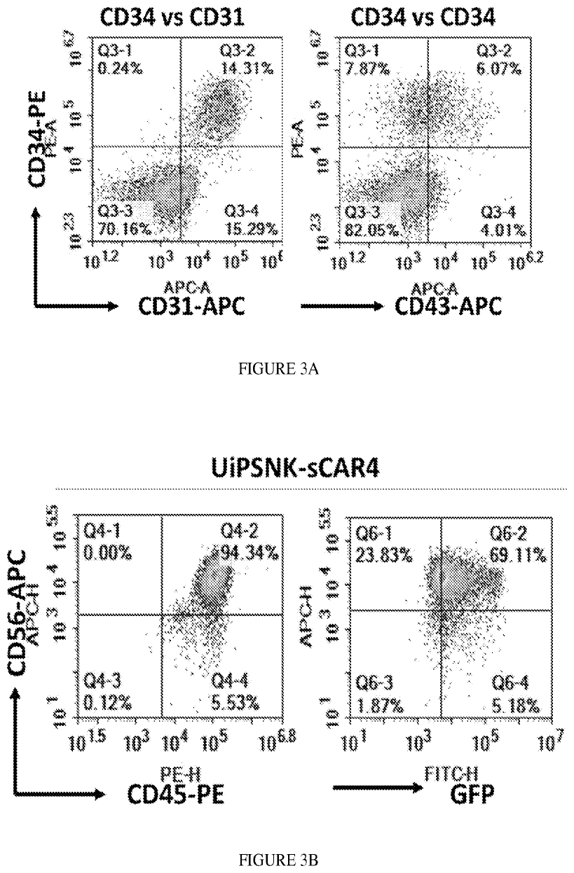

Successful derivation and function of sCAR-expressing iPSC-Derived NK cells. After confirming that the sCAR can be successfully expressed and function in NK92 cells the sCAR construct was expressed in human iPSCs. Here, a UCB-derived iPSC line (UiPSC) was used. The UiPSCs were transfected with PiggyBac-sCAR4, selected by zeocin and stable expression of sCAR was identified by GFP expression. Next, the UiPSCs were differentiated into mature NK cells using a two-stage differentiation process as previously described¹⁹⁻²². In stage I, sCAR-transfected iPSCs cultured with defined cytokines promote hematopoietic differentiation, as demonstrated by development of CD34⁺CD31⁺ and CD34⁺CD43⁺ hematopoietic progenitor cells. Next, these hematopoietic progenitor cells are differentiated into CD56⁺CD45⁺ NK cells that demonstrate stable CAR expression (GFP⁺) (FIGS. 3A-3B) and have normal phenotype with expression of CD56, NKG2A, NKG2C, NKG2D, NKp44, NKp46, KIRs, Fas, and TRAIL, as in previous studies^{19,20,23}.

FIGS. 3A-3B. Hematopoietic and NK cell differentiation in sCAR-expressing iPSCs. FIG. 3A shows normal hemato-endothelial cell differentiation showing CD34⁺CD31⁺ and CD34⁺CD43⁺ cells derived from sCAR-expressing iPSCs as seen in previous studies^{19,20}. FIG. 3B shows normal NK cell development from sCAR-expressing iPSCs showing >95% CD45+CD56+NK cells and >60% sCAR+CD56+NK cells. These iPSC-NK cells are expanded into a uniform >95% CD56+NK cell population as previously described^{19,20}.

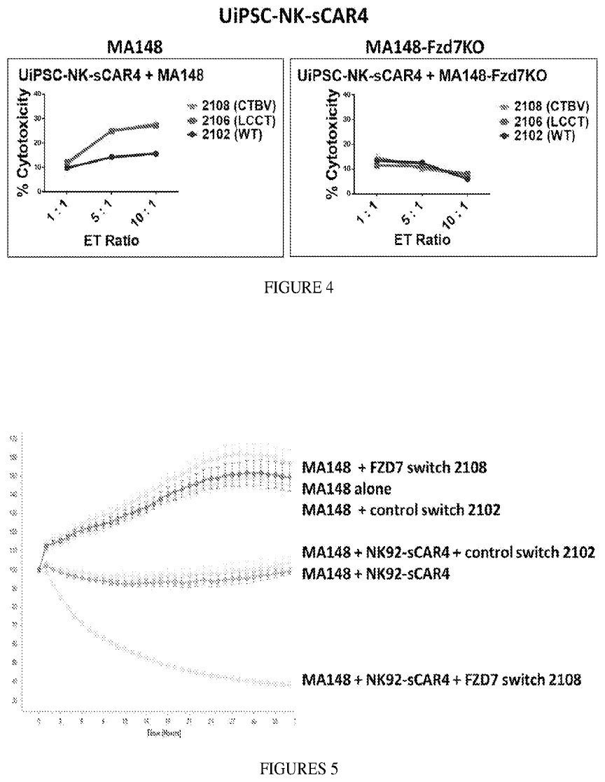

Anti-tumor activity of sCAR-expressing iPSC-NK cells. Similar to NK92-sCAR4 cells, iPSC-NK-sCAR4 cells were able to mediate antigen specific killing of tumor cell line MA148 in presence of 2 FDZ7-specific switches (2108-CTBV and 2106-LCCT), but not when FDZ7 was knocked out in MA148 cells (FIG. 4).

FIG. 4. sCAR mediated anti-tumor activity in iPSC-NK cells. iPSC-NK-sCAR4 cells were cocultured with target cells [either parental MA148 cells (left) or MA148-FDZ7 KO cells (right) in the presence of 1 nM of anti-FZD7 switches CTBV and LCCT or WT negative control switch (as in FIG. 1). These studies demonstrate effective sCAR+anti-FZD7-switch-mediated killing of the FZD7+ targets (left), but not the FZD7− targets (right).

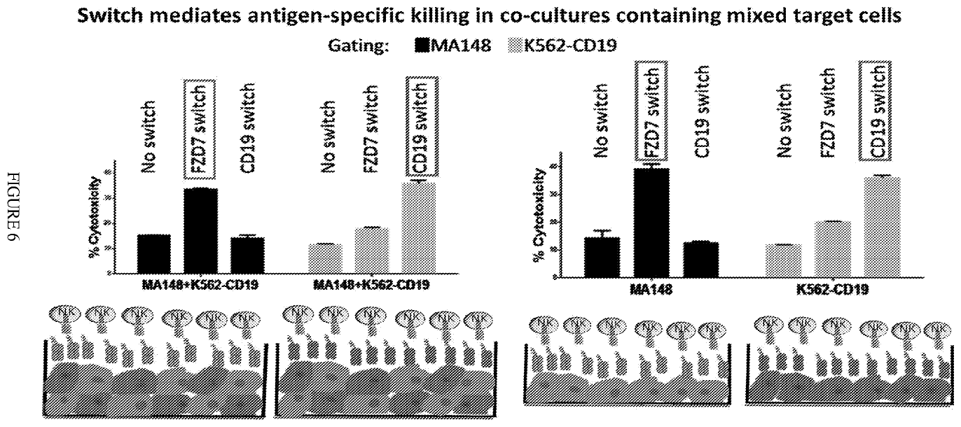

The switch-mediated killing of tumor cells by NK-sCAR-NK92 cells could be observed over a period of 35 hours directly under a fluorescence microscope. The killing could be measured quantitively using an IncuCyte-based assay (FIG. 5). Specifically, FIG. 5 shows an IncuCyte killing assay of MA148 cells by sCAR4-NK cells in the presence of Fzd7-specific switch CTBV (2108) and the control switch (2102).

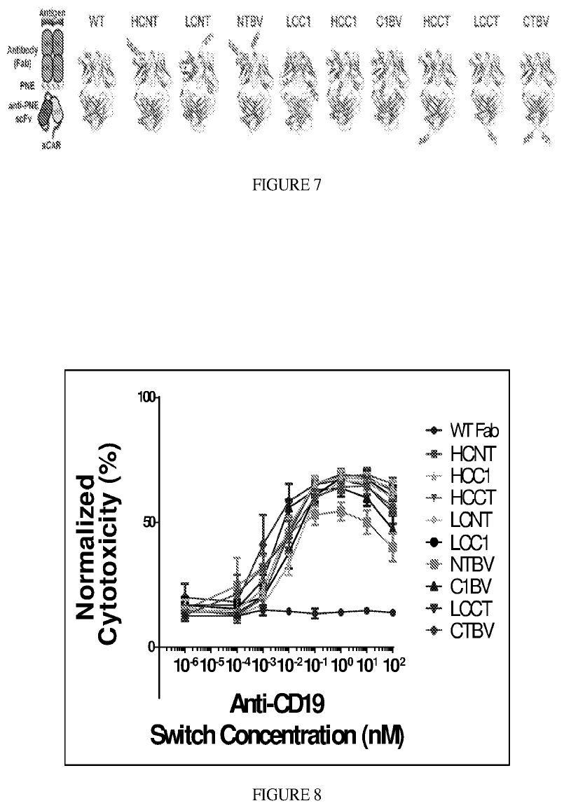

Finally, in cultures where cancer cells of different antigen specificity were mixed, a selective killing determined by the switch's antigen specificity was demonstrated (FIG. 6). Specifically, FIG. 6 shows switch-mediated antigen-specific killing of target cells in a mixed co-culture containing both MA148 and K562-CD19. Both switches induced specific killing in the mix culture (left) at the level comparable to that in separate cultures (right).

Together, this sCAR-NK cell strategy enables close control over CAR-mediated activity. Additionally, this system provides flexibility to target multiple antigens on tumor cells to potentially prevent antigen-loss escape variants that can lead to relapsed disease.

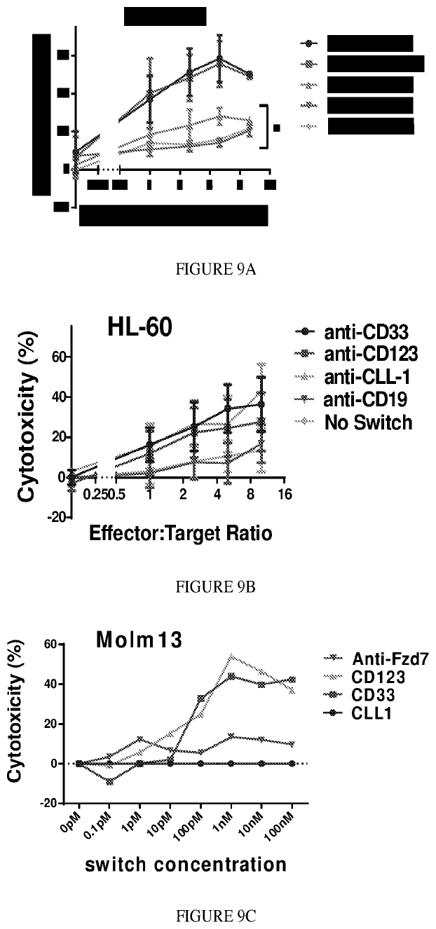

Test of anti-CD19 switches with NK92 expressing sCAR4. CAR-NK studies on B cell leukemia have been expanded from the initial one switch (namely LCNT) to a total of 10 anti-CD19 switches including a control switch lacking the PNE peptide (FIG. 7, Rodgers et al. 2016). The configurations, named according to the positions of the PNE engraftment, include: WT (Fab only without PNE), HCNT (heavy chain N-terminus (monovalent)), LCNT (light chain N-terminus (monovalent)), NTBV (N-terminus bivalent (both chains)), LCC1 (light chain C1). HCC1 (heavy chain C1), C1BV (C1 bivalently (both chains)), HCCT (heavy chain C-terminus), LCCT (light chain C-terminus), and CTBV (C-terminus bivalently (both chains)).

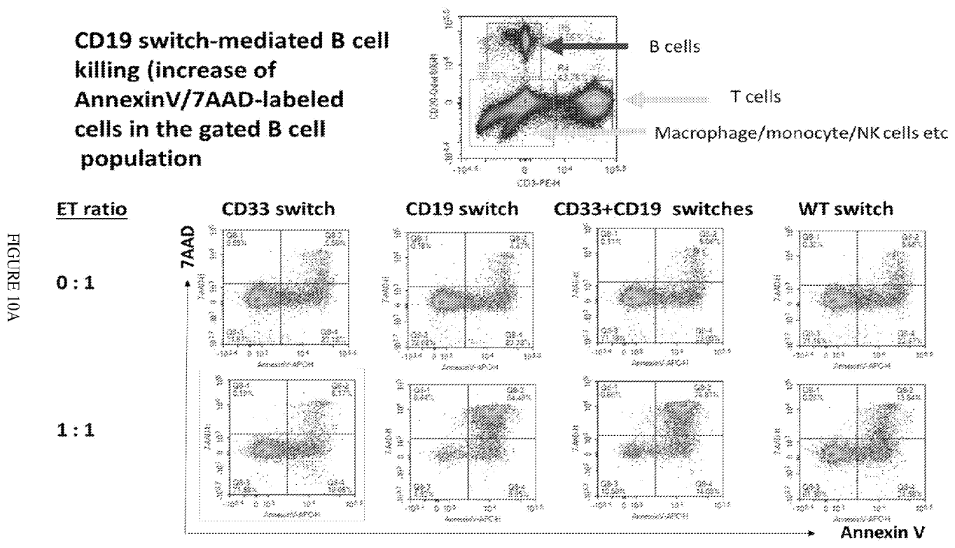

These CD19 switches have been tested with NK92-sCAR4 in an AnnexinV/7AAD killing assay (BIOLEGEND®). All switches mediated killing of Nalm6 cells by NK92-sCAR4 in a switch concentration-dependent manner, which is in a sharp contrast to the control WT switch (FIG. 8, n=3 or 4 per group+/−SEM). Specifically, FIG. 8 shows CAR4-NK92-induced killing of Nalm6 in the presence of the increased concentrations of anti-CD19 switches Killing EC₅₀ of each switch is calculated by Prizm, as shown in Table 1.

TABLE 1

| Anti-CD19 Switch | EC₅₀ (nM) | R² |
|---|---|---|
| WT Fab | N/A | — |
| HCNT | 0.0001947 | 0.7927 |
| HCC1 | 0.005599 | 0.9515 |
| HCCT | 0.006642 | 0.9454 |
| LCNT | 0.01647 | 0.947 |
| LCC1 | 0.01466 | 0.9078 |
| NTBV | 0.00812 | 0.5329 |
| C1BV | 0.002516 | 0.9033 |
| LCCT | 0.00721 | 0.924 |
| CTBV | 0.0008425 | 0.6742 |

Interestingly, these switches showed a slightly different relative kinetics in NK92 cell-mediated killing than that in sCAR-T cell-induced cytotoxicity as previously described by Rodgers et al 2016, suggesting that the relative efficacy of the switches determined in T cells cannot be directly translated for NK cells.

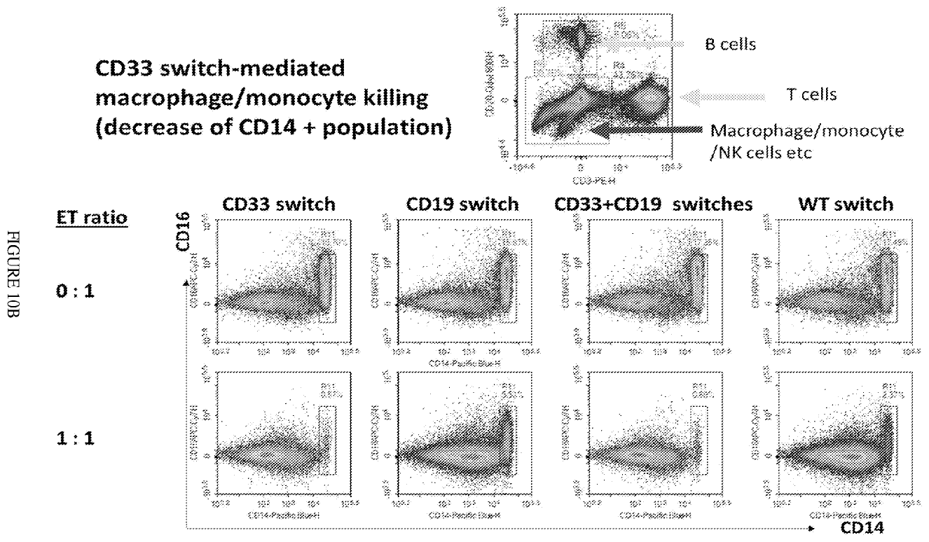

AML. The invention was extended to acute myeloid leukemia (AML) as an additional hematological malignancy to target. For this, switches were engineered to three AML-associated antigens, consisting of CD33, CD123, and CLL1. FIGS. 9A-9C show the surface expression of these antigens on AML cell lines Molm14 (FIG. 9A), HL60 (FIG. 9B) and Molm13, as well as the relative levels of killing mediated by these switches at different sCAR4-NK92 to Molm14 (FIG. 9A) or HL60 ratio (FIG. 9B), or at different concentrations of the switches (FIG. 9C). Results from HL60 (FIG. 9B) and Molm13 (FIG. 9C) seem to suggest a positive correlation between the sensitivity to killing and the level of antigen expressed on the target cells. Anti-CD19 and anti-Fzd7 switches were used here as a nonspecific switch control.

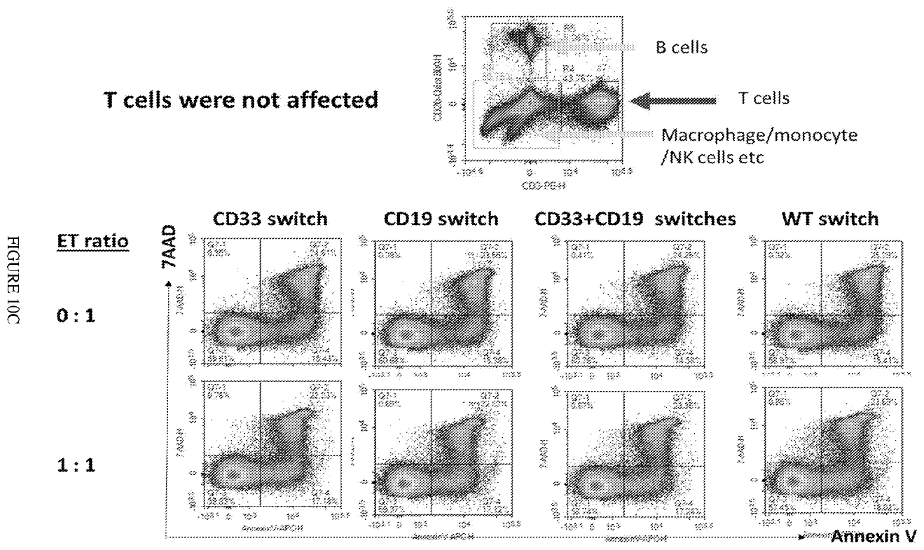

In the past, cells of different antigen specificity were artificially mixed and it was shown that the switches could mediate specific killing in these settings. To further illustrate switch-dependent antigen specificity in a more natural setting, commercial PBMCs (Precision for Medicine) of three independent donors were used and a killing assay in cocultures containing sCAR4-NK92 cells and either anti-CD33 or anti-CD19 switches or both were performed. Specific killing was observed of either macrophage/monocyte (express CD33) or B cells (express CD19) only when anti-CD33 or anti-CD19 switches, respectively, were present (FIGS. 10A-10C). The presence of the effector cells and the switches did not affect T cells as CD33 and CD19 are not expressed on these cells. Killing of macrophage/monocytes or B cells were minimum when a WT CD19 switch lacking PNE was used.

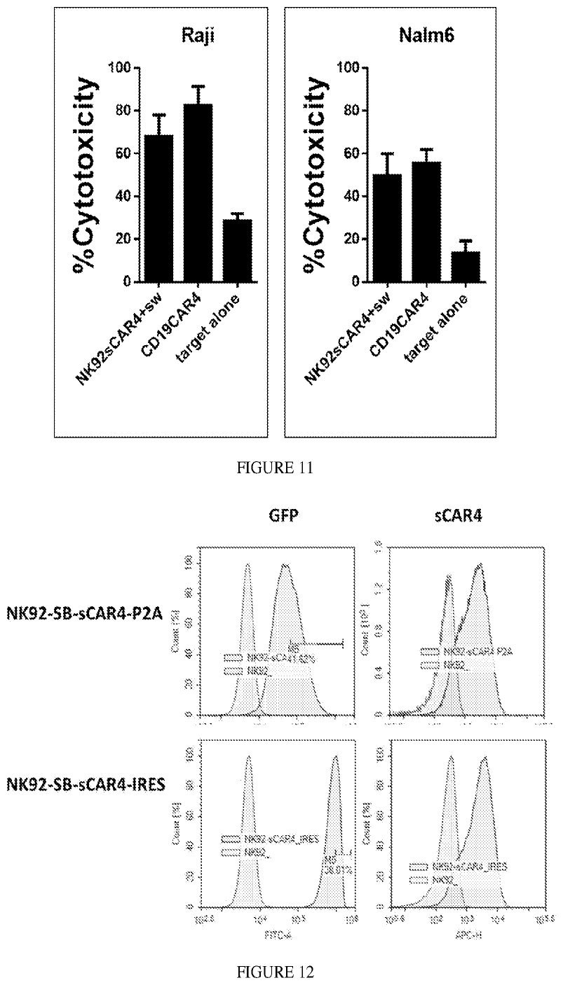

Comparison of sCAR with conventional CAR. To evaluate efficacy of the sCAR system comparing to the conventional CAR, two in vitro killing assays were carried out head-to-head (ET=1:1; n=3 per group+/−SD). In one assay, the coculture contained either Raji or Nalm6 B lymphoma cell line, the effector sCAR4-NK92 cells, and 10 pM anti-CD19 switch CTB V. In the second assay, CD19-CAR4-NK92 cells were directly cocultured with either Raji or Nalm6 cells. FIG. 11 shows the averaged cytotoxicity level. The cytotoxicity mediated by the switch and sCAR4 towards Raji is slightly lower than that mediated by the conventional CAR, whereas killing of Nalm6 is comparable for both CAR system. The similar comparisons will be also made using ovarian antigen Fzd7 and AML antigens as a target.

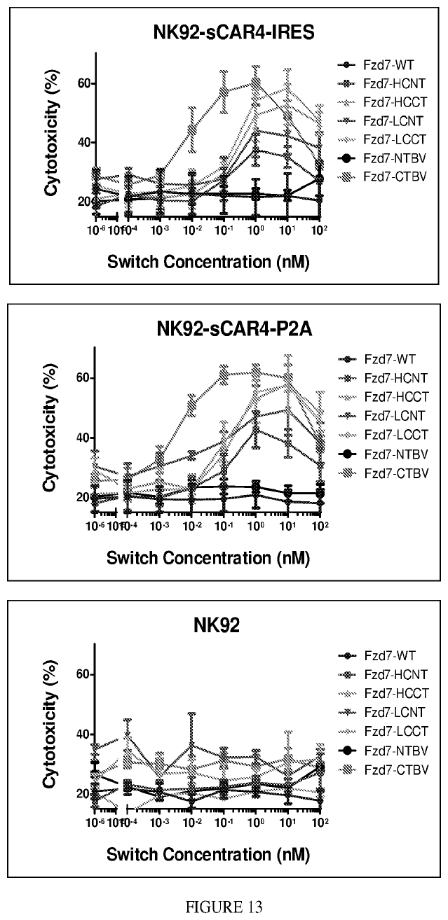

sCAR4-P2A-GFP construct. To better monitor expression of sCAR using GFP, a construct in which sCAR4 was fused in-frame to GFP with the cleavable peptide P2A in between (not shown) was engineered to replace the existing IRES fragment that facilitates a bi-cistronic expression of sCAR4 and GFP. NK92 cells transfected with sCAR4-P2A-GFP elucidated a similar level of in vitro cytotoxicity as the IRES-containing construct. FIG. 12 shows the level of GFP expression. Specifically, GFP and sCAR4 expression on NK92 cells transfected with either SB-sCAR4-P2A-GFP or SB-sCAR4-IRES-GFP. Comparison was made with WT NK92. Expression of sCAR4 was measured with Fc-PNE-AF647.

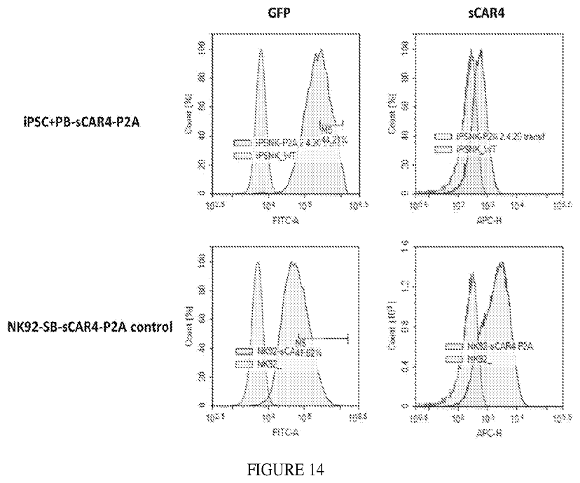

FIG. 13 shows a head-to-head comparison of in vitro killing of MA148 cells induced by NK92 cells expressing either sCAR4-P2AGFP and sCAR4-IRES-GFP in the presence of different anti-Fzd7 switches.

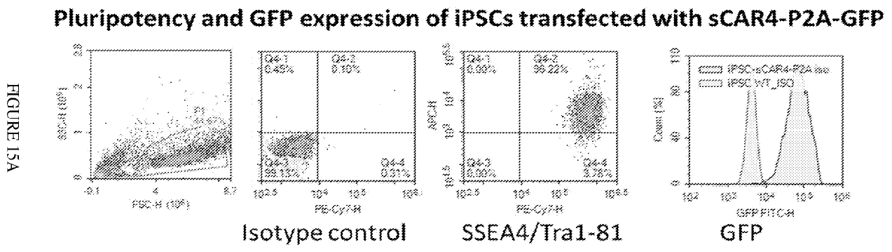

This construct was used to transfect iPSCs, aiming to regenerate mature NK cells. Expression of sCAR4 in sCAR4-P2A-GFP transfected iPSCs could be detected by FACS using Fc-NPE-AF647, although the level was lower than that on NK92 cells (FIG. 14).

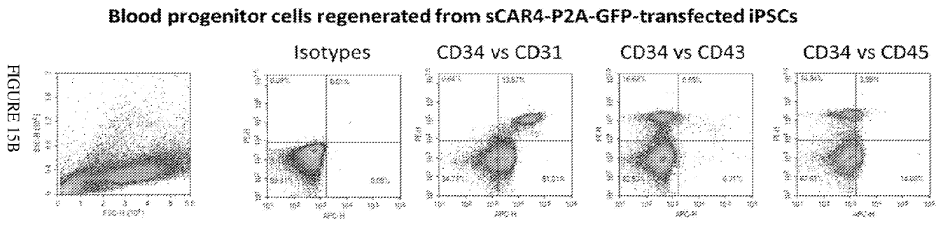

sCAR4-P2A-GFP-transfected iPSCs retained their pluripotency (FIG. 15A) and differentiated into hematopoietic progenitor cells (FIG. 15B). Mature NK cells are under regeneration and will be tested for their efficacy in killing tumor cells.

The results of this invention were surprising and advantageous. Prior to this invention, it had not been previously suggested or attempted to know how the described switchable PNE systems would work using NK cells. Although previous systems have been utilized for T cells, several factors set the sCAR-NK cell system of this invention apart from any sCAR-T cell system.

Prior to this invention, it was not clear that a switchable PNE system would work using NK cells due to the significant differences between NK cells and T cells. Expression of sCAR, either due to the density or duration on the cell surface, likely is different between NK and T cells, which can affect how the cells engage switches and target cells. Additionally, NK cells and T cells use different sets of surface receptors for activation, signaling regulation, and interaction with target cells. Therefore, with these different receptor and co-receptor interactions, it is not possible to predict how switches would engage NK cells based on how they work with T cells. Specifically, killing kinetics among different CD19 switches in sCAR4-NK92 cell-mediated Raji cell killing were different from that in T cell-engaged Nalm6 cell killing (Rodgers, et al 2016). As shown in this invention, the most efficient switches for T cells were not necessarily the most efficient ones for NK cells, and vice-versa. Whether or not this discrepancy is due to the influence of different surface receptor topology or sCAR expression levels between NK cells and T cells is not known.

In this invention optimization of the sCAR genetic vector for expression in iPSC-derived NK cells was also performed. Specifically, insulator sequences needed to be included in the expression plasmid (PiggyBac) to keep the sCAR gene from silencing, whereas such insulator was not needed for the sCAR expression in NK92 cell lines. With NK92 cells, the efficient expression of sCAR4 in NK92 cells was readily achieved by using a much smaller vector (SleepingBeauty system). Also, as a surrogate reporter protein, GFP expression faithfully represented the expression of sCAR4 on NK92 cells when the two proteins were expressed in a bicistronic manner mediated by an IRES fragment in the configuration of sCAR4-IRES-GFP. In contrast, GFP expression did not correctly indicate the expression of sCAR4 on iPSCs or iPSC-NK cells with the same configuration. To improve the expression system in iPSCs, a new construct was engineered where the IRES fragment was replaced with the P2A cleavage site. With this new construct, both GFP and sCAR4 was detected in the engineered iPSCs (FIG. 14).

Finally, NK cell-engaged switches, when confronting target cells, may behave differently than T cell-engaged switches in their strength of binding the antigen-bearing cells, therefore eliciting different levels of efficacy. In summary, by engineering sCAR4 into iPSC-derived NK cells, a completely different therapeutic cell product that has its own unique characteristics was made.

Overall, the novel iPSC-sCAR4-NK cell product of this invention provides significant advantages over any of the prior art, due to the intrinsic properties of NK cells as well as to the new attributes by the combination of NK with the sCAR system. The combination of the switch system and NK cells potentiates production of a true off-the-shelf (allogeneic) therapeutic approach, whereas the same combination using T cells would not. NK cells by themselves are allogenous effector cells, meaning that one batch of which can be expanded, stored and, used for a potentially unlimited number of patients. In contrast, T cells function as autologous cells, so need to be used in a patient-specific manner to avoid unwanted toxic side effects. By use of different switches, sCAR-expressing iPSC-derived NK cells need only to be engineered once and used both for different patients but also for different tumor targets. That is, this system means that sCAR-expressing iPSC-NK cells only need to be engineered once and will allow use in potentially any patient against any tumor antigen or virally-expressed antigen.

### REFERENCES

1. Bonifant C L, Jackson H J, Brentjens R J, Curran K J. Toxicity and management in CAR T-cell therapy. Mol Ther Oncolytics. 2016; 3:16011.
2. Fesnak A D, June C H, Levine B L. Engineered T cells: the promise and challenges of cancer immunotherapy. Nature Reviews Cancer. 2016; 16:566.
3. Brudno J N, Kochenderfer J N. Toxicities of chimeric antigen receptor T cells: recognition and management. Blood. 2016; 127(26):3321-3330.
4. Parkhurst M R, Yang J C, Langan R C, et al. T cells targeting carcinoembryonic antigen can mediate regression of metastatic colorectal cancer but induce severe transient colitis. Mol Ther. 2011; 19(3):620-626.
5. Morgan R A, Yang J C, Kitano M, Dudley M E, Laurencot C M, Rosenberg S A. Case report of a serious adverse event following the administration of T cells transduced with a chimeric antigen receptor recognizing ERBB2. Mol Ther. 2010; 18(4):843-851.
6. Gill S, Tasian S K, Ruella M, et al. Preclinical targeting of human acute myeloid leukemia and myeloablation using chimeric antigen receptor-modified T cells. Blood. 2014; 123(15):2343-2354.
7. Kenderian S S, Ruella M, Shestova 0, et al. CD33-specific chimeric antigen receptor T cells exhibit potent preclinical activity against human acute myeloid leukemia. Leukemia. 2015; 29(8):1637-1647.
8. Beyar-Katz O, Gill S. Novel Approaches to Acute Myeloid Leukemia
Immunotherapy. Clin Cancer Res. 2018; 24(22):5502-5515.
9. Beatty G L, Haas A R. Maus M V, et al. Mesothelin-Specific Chimeric Antigen Receptor mRNA-Engineered T Cells Induce Antitumor Activity in Solid Malignancies. Cancer Immunology Research. 2014; 2(2):112-120.
10. Cummins K D, Frey N, Nelson A M, et al. Treating Relapsed/Refractory (RR) AML with Biodegradable Anti-CD123 CAR Modified T Cells. Blood. 2017; 130(Suppl 1):1359-1359.
11. Diaconu I, Ballard B, Zhang M, et al. Inducible Caspase-9 Selectively Modulates the Toxicities of CD19-Specific Chimeric Antigen Receptor-Modified T Cells. Mol Ther. 2017; 25(3):580-592.
12. Rodgers D T, Mazagova M, Hampton E N, et al. Switch-mediated activation and retargeting of CAR-T cells for B-cell malignancies. Proc Natl Acad Sci USA. 2016; 113(4):E459-468.
13. Viaud S, Ma J S Y, Hardy I R, et al. Switchable control over in vivo CAR T expansion, B cell depletion, and induction of memory. Proc Natl Acad Sci USA. 2018; 115(46):E10898-E10906.
14. Cao Y, Rodgers D T, Du J, et al. Design of Switchable Chimeric Antigen Receptor T Cells Targeting Breast Cancer. Angew Chem Int Ed Engl. 2016; 55(26):7520-7524.
15. Raj D, Yang M H, Rodgers D, et al. Switchable CAR-T cells mediate remission in metastatic pancreatic ductal adenocarcinoma. Gut. 2018.
16. Morsut L, Roybal K T, Xiong X, et al. Engineering Customized Cell Sensing and Response Behaviors Using Synthetic Notch Receptors. Cell. 2016; 164(4):780-791.
17. Lim W A, June C H. The Principles of Engineering Immune Cells to Treat Cancer. Cell. 2017; 168(4):724-740.
18. Cho J H, Collins J J, Wong W W. Universal Chimeric Antigen Receptors for Multiplexed and Logical Control of T Cell Responses. Cell. 2018; 173(6):1426-1438.e1411.
19. Li Y, Hermanson D L, Moriarity B S, Kaufman D S. Human iPSC-Derived Natural Killer Cells Engineered with Chimeric Antigen Receptors Enhance Anti-tumor Activity. Cell Stem Cell. 2018; 23(2):181-192 e185.
20. Knorr D A, Ni Z, Hermanson D, et al. Clinical-Scale Derivation of Natural Killer Cells From Human Pluripotent Stem Cells for Cancer Therapy. STEM CELLS Translational Medicine. 2013; 2(4):274-283.
21. Bock A M, Knorr D, Kaufman D S. Development, expansion, and in vivo monitoring of human NK cells from human embryonic stem cells (hESCs) and induced pluripotent stem cells (iPSCs). J Vis Exp. 2013(74):e50337.
22. Hermanson D L, Bendzick L, Pribyl L, et al. Induced Pluripotent Stem Cell-Derived Natural Killer Cells for Treatment of Ovarian Cancer. STEM CELLS. 2016; 34(1):93-101.
23. Ni Z. Knorr D A, Bendzick L, Allred J, Kaufman D S. Expression of Chimeric Receptor CD4ζ by Natural Killer Cells Derived from Human Pluripotent Stem Cells Improves In Vitro Activity but Does Not Enhance Suppression of HIV Infection In Vivo. STEM CELLS. 2014; 32(4):1021-1031.
24. June C H, Sadelain M. Chimeric Antigen Receptor Therapy. New England Journal of Medicine. 2018; 379(1):64-73.
25. P S Woll, C H Martin, J S Miller, D S Kaufman. Human embryonic stem cell-derived NK cells acquire functional receptors and cytolytic activity. J Immunol, 175 (2005), pp. 5095-5103.
26. Deng X, Antitumor activity of NKG2D CAR-T cells against human colorectal cancer cells in vitro and in vivo. Am J Cancer Res. 2019; 9(5): 945-958.

What is claimed is:

1. A natural killer (NK) cell engineered with a switchable chimeric antigen receptor (sCAR).

2. The NK cell of claim 1, wherein the sCAR comprises an antibody scFv region specific for binding to a peptide neoantigen epitope (PNE).

3. The NK cell of claim 2, wherein the sCAR further comprises an NKG2D transmembrane domain, 2B4 co-stimulatory domain, and CD3ζ chain.

4. The NK cell of claim 3, further comprising a switch bound to the sCAR, wherein the switch comprises a PNE fused to an anti-cancer or anti-virus antibody Fab region specific for binding to a cancer antigen or virus antigen.

5. The NK cell of claim 5, wherein the cancer antigen is CD19 or Frizzled 7.

6. The NK cell of claim 1, wherein the NK cell is derived from a human induced pluripotent cell.

7. A method of treating a cancer or a virus in a subject comprising administering to a subject in need thereof an effective amount of a natural killer (NK) cell engineered with a switchable chimeric antigen receptor (sCAR) activated against an antigen of the cancer or the virus.

8. The method of claim 7, wherein the sCAR comprises an antibody scFv region specific for binding to a peptide neoantigen epitope (PNE).

9. The method of claim 8, wherein the sCAR further comprises an NKG2D transmembrane domain, 2B4 co-stimulatory domain, and CD3ζ chain.

10. The method of claim 9, wherein the sCAR is activated by being bound to a switch, wherein the switch comprises a PNE fused to an anti-cancer or anti-virus antibody Fab region specific for binding to the cancer antigen or virus antigen, respectively.

11. The method of claim 10, wherein the cancer antigen is CD19 or Frizzled 7.

12. The method of claim 7, wherein the NK cell is allogenic.

13. The method of claim 7, wherein the cancer is refractory.

14. The method of claim 7, wherein the cancer is hemotologic or a solid tumor.

15. The method of claim 14, wherein the tumor is lymphatic or ovarian.

16. The method of claim 7, wherein the method further comprises administration of a therapeutic amount of monoclonal antibody therapy against the cancer or virus.

17. A pharmaceutical composition comprising the NK cell of claims 1-6.

18. A method of manufacturing a natural killer (NK) cell, comprising:
engineering a NK cell to display a transmembrane protein comprising a switchable chimeric antigen receptor (sCAR); and
storing the engineered NK cell for later activation of the sCAR.

19. The method of claim 18, wherein the sCAR comprises an antibody scFv region specific for binding to a peptide neoantigen epitope (PNE).

20. The method of claim 19, wherein the sCAR further comprises an NKG2D transmembrane domain, 2B4 co-stimulatory domain, and CD3ζ chain.

21. The method of claim 20, further comprising:
activating the sCAR by binding the sCAR to a switch, wherein the switch comprises a PNE fused to an anti-cancer or anti-virus antibody Fab region specific for binding to a cancer antigen or virus region, respectively.

22. The method of claim 21, wherein the cancer antigen is CD19 or Frizzled 7.
